(19) United States
(12) Patent Application Publication
(10) Pub. No.: US 2023/0354879 A1
(43) Pub. Date: Nov. 9, 2023

(54) CHEMICAL COMPOSITION FOR VAPING DEVICE

(71) Applicant: Etan Barak, North Miami, FL (US)

(72) Inventor: Etan Barak, North Miami, FL (US)

(21) Appl. No.: 18/076,536

(22) Filed: Dec. 7, 2022

Related U.S. Application Data
(60) Provisional application No. 63/339,067, filed on May 6, 2022.

(51) Int. Cl. A24B 15/167

(52) U.S. Cl. CPC A24B 15/167

(57) ABSTRACT

A chemical composition suitable for use in a vaping device, smoking, and/or other forms of consumption including a constituent group of ingredients is provided. The chemical composition of the constituent group of ingredients is operable for use by a male only user, a female only user, and to be shared by both a male user and a female user. The chemical composition is different each of the male only user, the female only user, and that intended to be shared by both the male user and the female user.

### CROSS-REFERENCE TO RELATED APPLICATIONS

The present application claims priority to U.S. Provisional Patent Application No. 63/339,067 filed May 6, 2022, entitled “COMPOSITION FOR VAPING DEVICE” which is hereby incorporated by reference in its entirety.

### FIELD

The present disclosure relates to a chemical composition, and more particularly to a chemical composition for use in smoking, vaping, aromatizing, and/or the like, with various desirable effects, such as relaxation, energizing, increased sensitivity, male and/or female enhancement, vasodilation, and the like.

### BACKGROUND

This section provides background information related to the present disclosure which is not necessarily prior art. There are various chemical compositions that cause different effects in the human body and experience. Generally, scientists and businesses research and develop chemical compositions, compounds, solutions, or mixtures that address specific diseases and ailments. For example, acetaminophen for pain, albuterol for asthma, chamomile tea for sleep and relaxation, and the like. While many of these conventional chemical compositions, compounds, solutions, and mixtures, hereinafter “chemical composition”, may have more than one effect or purpose, they are not developed for multi-effect and/or multipurpose use, much less for their multi-effect and/or multipurpose use when shared by two or more persons. Especially, when one of the intended purposes may include a sexual activity. Therefore, there is need in the art to develop chemical compositions that are suitable for use in a smoking, vaping device, aromatizing, and the like, which can achieve one or more effects and/or serve one or more purposes, by a person, or persons, engaged in relaxation or collaborative activities, which may include collaborative sexual activities.

### SUMMARY OF THE INVENTION

This section provides a general summary of the disclosure and is not a comprehensive disclosure of its full scope or all of its features.

The present disclosure relates to a chemical composition having various desirable effects and purposes for use in smoking, vaping device, aromatizing, and/or the like. More specifically, this section provides a general summary of a chemical composition for use in at least a vaping device. The chemical compositions having various desirable effects, including but not limited to relaxation, energizing, increased sensitivity, vasodilation, and enhancement, each of the compositions being specifically tailored to a male only user, a female only user, and shared between male and female users. For example, in the case of vasodilation the proposed chemical composition(s) causes the muscular walls of the blood vessels to relax and thereby widen, which in turn causes a decrease in blood pressure. That is, vasodilation causes a decrease in Systemic Vascular Resistance (SVR) and an increase in blood flow, resulting in a reduction in blood pressure. Therefore, vasodilation can be considered a mechanism for enhancing blood flow to areas of the body that are lacking oxygen and/or nutrients.

In some embodiments, a chemical composition suitable for use in a vaping device includes a constituent group of ingredients. The chemical composition of the constituent group of ingredients is operable for use by a male only user, a female only user, and to be shared by both a male user and a female user. The chemical composition is different for the male only user, the female only user, and the one that is intended to be shared by both male and female users.

In various embodiments, a chemical composition intended to be shared by both male and female users is operable to produce two or more different effects and/or has two or more different purposes. One of these purposes may be a sexual activity.

In various embodiments, a chemical composition intended to be shared by both male and female users is different from the chemical compositions intended to be used by a male only user and the chemical composition intended to be used by a female only user.

In various embodiments, the composition of the chemical composition intended for a female only user includes one or more active ingredient(s) that the chemical composition intended for a male only user does not contain.

In various embodiments, the composition of the chemical composition intended for a male only user is different from the composition of the chemical intended for a female only user. For example, the composition of the chemical composition intended for male only users includes a greater amount of an active ingredient than the chemical composition intended for female only users.

In various embodiments, the composition of the chemical composition intended for a male only user includes one or more active ingredient(s) that the chemical composition intended for a female only user does not contain.

In various embodiments, the chemical composition having attributes tailored for a male only user is preferred over the chemical composition having attributes tailored for a female only user for a user who is biosimilar to a male user.

In various embodiments, the chemical composition having attributes tailored for a female only user is preferred over the chemical composition having attributes tailored for a male only user for a user who is biosimilar to a female user.

In various embodiments, the same active ingredient is used in chemical compositions intended for a male only user and a female only user. In some embodiments, the amount of the active ingredient used by in the chemical composition intended for a male only user is less than the amount of active ingredient used in the chemical composition intended for a female only user. For example, the amount of one or more of: 3-Carene, alpha-Pinene, beta-Pinene, Camphene, Fenchone, Geraniol, Hexahydrothymol, trans-Caryophyllene, Linalool, Nerol, and Terpineol used in the chemical composition intended for a male only user is less than the amount used in the chemical composition intended for a female only user.

In various embodiments, the chemical composition includes at least one active ingredient. In some embodiments, the chemical composition includes one or more of a pharmaceutically acceptable preservative, excipient, and additive. In some embodiments, the chemical composition also includes a pharmaceutically acceptable pH adjusting agent. For example, the chemical composition includes one or more of an alkali, an organic acid, an inorganic acid, and the like. In some embodiments, the chemical composition also includes at least one pharmaceutically acceptable solvent, which may be an alcohol based solvent and/or a water based solvent.

In various embodiments, the chemical composition includes a constituent group of ingredients. In some embodiments, the constituent group of ingredients includes a cannabinol based product, For example, hexahydro cannabinol. In some embodiments, the constituent group of ingredients includes a cannabinol based product. For example, hexahydro cannabinol. In some embodiments, the constituent group of ingredients incudes a limonene based product. For example one or more of the following: 3-Carene, Alpha-Pinene, Alpha-Humulene, Ashwagandha, Beta-Myrcene, Beta-Pinene, Camphene, Caryophyllene oxide, Eucalyptol, Fenugreek seed extract, Fenchone, Gamma-Terpinene, Geraniol, Geranyl acetate, Hexahydrothymol, Horny goat weed extract, Trans-Caryophyllene, (R)-(+)-Limonene, Linalool, Nerol Terpineol, Tribulus Terrestre's, Terpinolene, Maca root extract, and the like.

In some embodiments, a vaping device includes a chemical composition suitable for use in vaping. The chemical composition includes a constituent group of ingredients. The chemical composition of the constituent group of ingredients is operable for use by a male only user, a female only user, and to be shared by both a male and female users. The chemical composition is different each of the male only user, the female only user, and that intended to be shared by both the male and female users.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS AND TABLES

The features and advantages of the present disclosure will be more fully described in, or rendered obvious by, the following detailed description of the preferred embodiments, which are to be considered together with the accompanying drawings, wherein like numbers refer to like parts and further, wherein:

FIG. 1 is a perspective view of a portable atomizer, in accordance with some embodiments, described herein; and

FIG. 2 is a perspective view of a pen-style vape, in accordance with some embodiments described herein.

TABLE 1 describes a chemical composition having attributes tailored for enhancement of a male only user's vaping experience, in accordance with some embodiments described herein; and

TABLE 2 describes a chemical composition having attributes tailored for enhancement of a female only user's vaping experience, in accordance with some embodiments described herein.

### DETAILED DESCRIPTION

Any reference to “invention” within this document is a reference to an embodiment of a family of inventions, with no single embodiment including features that are necessarily included in all embodiments, unless otherwise stated. Furthermore, although there may be references to “advantages” provided by some embodiments, other embodiments may not include those same advantages, or may include different advantages. Any advantages described herein are not to be construed as limiting to any of the claims.

As used herein, relational terms, such as “first” and “second,” “top” and “bottom,” “forward” and “rearward,” and the like, may be used solely to distinguish one entity or element from another entity or element without necessarily requiring or implying any physical or logical relationship or order between such entities or elements.

Specific quantities, dimensions, spatial characteristics, chemical compositional characteristics, and performance characteristics may be used explicitly or implicitly herein, but such specific quantities are presented as examples only and are approximate values unless otherwise indicated. Discussions and depictions pertaining to these, if present, are presented as examples only and do not limit the applicability of other characteristics, unless otherwise indicated.

In describing preferred and alternate embodiments of the technology described herein, specific terminology is employed for the sake of clarity. The technology described herein, however, is not intended to be limited to the specific terminology so selected, and it is to be understood that each specific element includes all technical equivalents that operate in a similar manner to accomplish similar functions.

Example embodiments will now be described more fully with reference to the accompanying drawings. Specific details are set forth such as examples of specific components and methods to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known device structures and/or chemical composition structures are not described in detail.

In various embodiments, a chemical composition, and more particularly one or more chemical compositions having various desirable benefits, effects, and purposes that is suitable for use with a vaping device is provided. In some embodiments, the chemical composition is suitable for topical application, lacing cigarettes, mixing in food or drinks, vaping and/or smoking with and/or without the use of a vaping device or a cigarette, and the like, is provided.

The proposed chemical composition may be used with any suitable vaping device known to a person of ordinary skill in the art, including those vaping devices having permanent cartridges, replaceable cartridges, and refillable cartridges. These vaping devices may include but are not limited to ceramic vaporizers, convection vaporizers, direct coil vaporizers, heated plate vaporizers, and ultrasonic vaporizers. These vaporizers are configured to operate over a wide temperate range. For example, between about 160 degrees Celsius to about 240 degrees Celsius, with the most common range being between about 190 degrees Celsius to about 235 degrees Celsius. Generally, between about 160 degrees Celsius and about 180 degrees Celsius is considered the lower temperature range for vaping, while between about 180 degrees Celsius to about 200 degrees Celsius is considered the mid temperature range for vaping, and between about 200 degrees Celsius and about 235 degrees Celsius being considered the high temperature range for vaping.

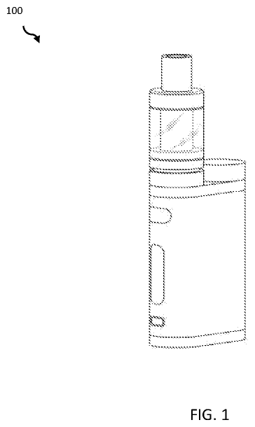

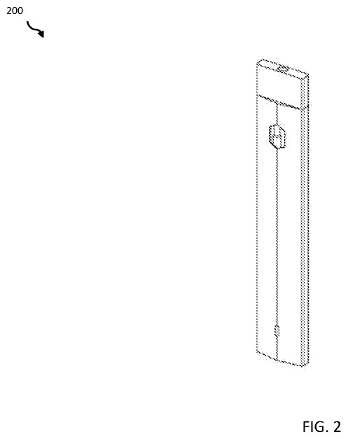

In various embodiments, the method of dispensing the chemical composition comprises a portable vape atomizer 100 as shown in FIG. 1. A portable vape atomizer 100 is generally larger and more powerful than the more common pen-style vape depicted in FIG. 2. The portable vape atomizer 100 is fitted with a more robust power source than the pen-style vape, and the powerful design makes the portable vape atomizer 100 the best portable vape device for dry herb vaping. The portable vape atomizer 100 is often fitted with temperature control that allows the user to select the precise temperature that the chemical composition is vaped at. The ability to select the precise temperature is key to consistent vaping, no matter how dry or moist the chemical composition or herb is.

In various embodiments, the method of dispensing the chemical composition comprises a vape pen 200 as shown in FIG. 2. A vape pen 200 is a handheld device consisting of a battery, a heating element, and a cartridge filled with a cannabis concentrate. Vape pens 200 vaporize rather than burn cannabis. In various embodiments, the vape pen 200 comprises a cartridge or container for the chemical composition that will be vaporized. The container may be a tank or cartridge made of steel, plastic, glass, and the like. The container may be a pre-filled cartridge filled with distillate or concentrate that is used until empty and then disposed of or recycled. In some instances, the cartridges may be refillable tank. In various embodiments, the vape pen 200 comprises a heating element (atomizer) that vaporizes the chemical composition. The atomize is powered by a battery. For example, a rechargeable lithium-ion battery that powers the vape pen 200 and heats the concentrate by means of the atomizer. In some instances, the heating of the chemical composition is activated with the press of a button. Alternatively, the heating of the chemical composition many be activated by the user inhaling through a mouthpiece through which the chemical composition vapor is inhaled. A USB charger may be used, and/or included, with the vape pen 200 to charge the rechargeable lithium-ion battery. In some instances, a disposable vape pen 200 may comprise a pre-charged disposable battery.

In various embodiments, the chemical composition includes a constituent group of ingredients. In some embodiments, the constituent group of ingredients includes a cannabinol based product. For example, hexahydro cannabinol. In some embodiments, the constituent group of ingredients incudes a limonene based product. For example one or more of the following ingredients: 3-Carene, Alpha-Pinene, Alpha-Humulene, Ashwagandha, Beta-Myrcene, Beta-Pinene, Camphene, Caryophyllene oxide, Eucalyptol, Fenugreek seed extract, Fenchone, Gamma-Terpinene, Geraniol, Geranyl acetate Hexahydro cannabinol, Hexahydrothymol, horny goat weed extract trans-Caryophyllene, (R)-(+)-Limonene, Linalool, Nerol Terpineol, Tribulus Terrestre's, Terpinolene, Maca root extract, and the like. Each one of these ingredients adds a characteristic that, when the chemical composition is consumed by a user, results in one or more effects. In other words, each ingredient or combinations of ingredients adds one or more desired effects to the chemical composition and/or is included for one or more purposes, which the user may or may not be immediately aware of at the time of vaping. In other words, an effect is caused based on one or more of the ingredients and/or the combinations thereof.

In various embodiments, the amounts, combinations, purity, quality, and proportions of ingredients in the chemical composition can be altered to modify the effects and purpose of the chemical composition. For example, some of the ingredients in the proposed chemical composition(s) are purported to have a variety of health benefits. In some instances, these health benefits have been established, while in others they have been purported but have yet to be established. For example, some of the reported health benefits of Ashwagandha include stress relief and anxiety treatment. While fenugreek reportedly lowers blood sugar levels, boosts testosterone levels, and increases milk production in breastfeeding mothers. While the maca root reportedly increases libido, reduces erectile dysfunction, blood pressure level, and sun damage. The maca root also reportedly increases energy, endurance, and fertility, and fights the adverse effects of free radicals, while improving the mindset of the user, that is their mood.

In contrast to, or in addition to, the many purported health benefits of some of the ingredients in the proposed chemical composition(s) some of the ingredients are included for one or more purposes, such as to enhance the taste, aroma, flavor, scent, texture, sweetness, and the like, of the chemical composition. For example, Nerol is a monoterpenoid alcohol found in many essential oils such as lemongrass and hops. It was originally isolated from neroli oil, hence its name. This colorless liquid is used in perfumery. Like geraniol, Nerol has a sweet rose odor, but it is considered to be fresher.

The amounts, purity, quality, and proportions of the ingredients in the chemical composition affect the qualities and effects of vaping, smoking, and/or otherwise consuming the chemical composition. In various embodiments, one or more of the amounts, purity, quality, and proportions of the ingredients in the chemical composition are varied within predefined limits.

In various embodiments, the chemical composition is base, at least in part, upon a constituent group of ingredients, which may or may not include active ingredients. The constituent group of ingredients may form the basis of any chemical composition that can be further modified for a particular group of users. For example, the constituent group of ingredients may be further modified to make a chemical composition that is intended for female only users. Likewise, the constituent group of ingredients may be further modified to make a chemical composition that is intended for female only users. Alternatively, the constituent group of ingredients may be further modified to made chemical composition that is intended to be shared by both male and female users. In some embodiments, the constituent group of ingredients is intended to be shared by both male users and female users without further modification. In some embodiments, the constituent group of ingredients contributes minimum or negligible desired effects.

In various embodiments, the constituent group of ingredients comprises one or more of the following ingredients: 3-Carene, Alpha-Pinene, Alpha-Humulene, Ashwagandha, Beta-Myrcene, Beta-Pinene, Camphene, Caryophyllene oxide, Eucalyptol, Fenugreek seed extract, Fenchone, Gamma-Terpinene, Geraniol, Geranyl acetate Hexahydro cannabinol, Hexahydrothymol, horny goat weed extract trans-Caryophyllene, (R)-(+)-Limonene, Linalool, Nerol, Terpineol, Tribulus Terrestre's, Terpinolene, trans-Caryophyllene, Maca root extract, and the like.

In various embodiments, the chemical composition intended for male only users comprises one or more of the following ingredients: hexahydro cannabinol, 3-Carene, Alpha-Pinene, Alpha-Humulene, Beta-Myrcene, Beta-Pinene, Camphene, Caryophyllene oxide, Eucalyptol, Fenchone, Gamma-Terpinene, Geraniol, Geranyl acetate, Hexahydrothymol (R)-(+)-Limonene, Linalool, Nerol, Terpineol, Terpinolene, and trans-Caryophyllene. In some embodiments, the chemical composition intended for male only users comprises all of the aforementioned ingredients.

In various embodiments, the chemical composition intended for female only users comprises one or more of the following ingredients: hexahydro cannabinol, 3-Carene, Alpha-Pinene, Alpha-Humulene, Beta-Myrcene, Beta-Pinene, Camphene, Caryophyllene oxide, Eucalyptol, Fenchone, Geraniol, Hexahydrothymol, (R)-(+)-Limonene, Linalool, Nerol, Terpineol, and trans-Caryophyllene, In some embodiments, the chemical composition intended for female only users comprises all of the aforementioned ingredients.

In some embodiments, the chemical composition intended to be shared by both male and female users comprises one or more of the following ingredients: 3-Carene, Alpha-Pinene, Alpha-Humulene, Ashwagandha, Beta-Myrcene, Beta-Pinene, Camphene, Caryophyllene oxide, Eucalyptol, Fenugreek seed extract, Fenchone, Gamma-Terpinene, Geraniol, Geranyl acetate Hexahydro cannabinol, Hexahydrothymol, horny goat weed extract trans-Caryophyllene, (R)-(+)-Limonene, Linalool, Nerol Terpineol, Tribulus Terrestre's, Terpinolene, Maca root extract, and the like.

Each of the active ingredients in the chemical composition, either alone or in combination with one or more other active ingredients, contributes one or more desired effects to the chemical composition. In some embodiments, the desirable effect may be an underlying health benefit(s), which may or may not be immediately apparent to the user.

Each of the active ingredients in the chemical composition, either alone or in combination with one or more other active ingredients, contributes one or more desired purposes to the chemical composition. In some embodiments, the desirable purpose may be a characteristic of the chemical composition. For example, the composition of the chemical composition may be configured to promote weight loss or as a sleep aid. In some embodiments, the chemical composition is based, at least in part, on the effects that user desires the chemical composition to produce.

In various embodiments, one or more of the active ingredients, may add a characteristic to the chemical composition, such as flavor, aroma, texture, volume, and the like, when the chemical composition is consumed by the user.

In various, the amounts, combinations, purity, quality, and proportions of the active ingredient(s) in the chemical composition are modified to alter the effects and purpose of the chemical composition when consumed by the user.

In various embodiments, to generate a chemical composition appropriate for consumption in consideration of the desired effects and purpose, the attributes of the individual user may also be taken into consideration when determining the appropriate and optimum chemical composition for that intended user. That is, one or more active ingredients are added, removed, combined, and/or mixed in the composition of the chemical composition in consideration of the attributes of the individual user. For example, the composition of the chemical composition may be tailored to one or more of the users age, activity level, Body Mass Index (BMI), medical history, illness, and weight. Further, the composition of the chemical composition may be tailored to whether the chemical composition is intended to be used with alcohol, during pregnancy, by nursing mothers, and the like.

TABLE 1 describes a chemical composition having attributes that have been tailored for enhancement of a male user's vaping experience. That is, TABLE 1 describes a first enhanced chemical composition intended for a male only user.

TABLE 1

| | | | | Percentage | |
|---|---|---|---|---|---|
| | | Variance | | Variance | |
| Ingredient | Mg/G | (+/−) | Percentage | (+/−) | Effect |
| Hexahydro | 800 | 160 | 80.00% | 16.00% | Promotes Relaxation, |
| cannabinol | | | | | Heighten Sensitivity |
| | | | | | to Touch, and |
| | | | | | Increased Intensity of |
| | | | | | Feelings |
| (R)-(+)-Limonene | 13.66 | 2.73146 | 1.37% | 0.27% | Elevate Mood by |
| | | | | | Increasing Serotonin, |
| | | | | | Dopamine, |
| | | | | | Norepinephrine, and |
| | | | | | GABA concentrations |
| | | | | | in the Brain |
| Hexahydrothymol | 3.15 | 0.63067 | 0.32% | 0.06% | Ergogenic: may |
| | | | | | enhance physical |
| | | | | | performance and |
| | | | | | stamina. |
| alpha-Pinene | 2.79 | 0.55861 | 0.28% | 0.06% | Increase Feeling of |
| | | | | | Energy |
| trans- | 2.62 | 0.52487 | 0.26% | 0.05% | Reduce Stress |
| Caryophyllene | | | | | |
| Linalool | 2.56 | 0.51129 | 0.26% | 0.05% | Induce Body and |
| | | | | | Mind Relaxation - |
| | | | | | May increase Blood |
| | | | | | Flow through |
| | | | | | Vasodilation |
| 3-Carene | 2.48 | 0.49693 | 0.25% | 0.05% | Improve Mental State - |
| | | | | | May Improve |
| | | | | | Attentiveness |
| beta-Pinene | 2.32 | 0.46346 | 0.23% | 0.05% | Increase Alertness |
| Eucalyptol | 2.14 | 0.42829 | 0.21% | 0.04% | Promote Calmness |
| beta-Myrcene | 2.08 | 0.41665 | 0.21% | 0.04% | Enhance |
| | | | | | Bioavailability |
| Geraniol | 1.74 | 0.34722 | 0.17% | 0.03% | Enhance Relaxation - |
| | | | | | Vasodilation |
| Terpineol | 0.81 | 0.16279 | 0.08% | 0.02% | Reduce Anxiety - |
| | | | | | May increase Blood |
| | | | | | Flow through |
| | | | | | Vasodilation |
| Nerol | 0.74 | 0.14865 | 0.07% | 0.01% | Vasodilation |
| Gamma-Terpinene | 0.51 | 0.10137 | 0.05% | 0.01% | Reduce Tension and |
| | | | | | Increase Feelings |
| | | | | | of Relaxation |
| Fenchone | 0.31 | 0.061 | 0.03% | 0.01% | Aromatic Balance |
| Caryophyllene | 0.24 | 0.0475 | 0.02% | 0.00% | CB2 Receptor |
| oxide | | | | | Agonist for |
| | | | | | Enhancement of |
| | | | | | Entourage Effect |
| alpha-Humulene | 0.14 | 0.0288 | 0.01% | 0.00% | Enhance |
| | | | | | Bioavailability |
| Terpinolene | 0.06 | 0.01182 | 0.01% | 0.00% | Promotion of |
| | | | | | Relaxation |
| Camphene | 0.05 | 0.01034 | 0.01% | 0.00% | Reduce Stress |
| Geranyl acetate | 0.03 | 0.006 | 0.00% | 0.00% | Reduce Stress and |
| | | | | | Uplift Mood |

In various embodiments, one or more of these active ingredients are included for their purported effect and/or distinctive, pervasive, and pleasant smell, aroma and/or purported medical benefits. For example, referring once more to TABLE 1 and TABLE 2, Delta 3 Carene is purported to relieve inflammation related to arthritis or fibromyalgia. It also plays a major role in boosting bone health, thereby benefitting people with osteoporosis, osteoarthritis, and other bone related diseases. Delta 3 Carene is also said to promote mental focus and concentration. Pinene is one of the most commonly found terpenes in cannabis and is also one of the most frequency used and well understood terpenes. Beta-prime has been shown to be an effective anti-inflammatory, a bronchodilator, pain reliever, anxiety-reliever, and even a tool to battle short-term memory impairment. Eucalyptol has been shown to relieve sinus and nasal congestion and improve breathing issues like asthma. Fenchone is purported to have anti-inflammatory, antimicrobial, properties decrease the number of inflammatory cells during wound healing. Trans-caryophyllene has been shown have antibacterial, soothing, antimicrobial, and antioxidant properties. Nerol has a citrusy, floral, and slightly bitter. β-Myrcene contained within the cannabis plant may improve sleep, reduce pain, promote relaxation, enhance a person's mood, relieve anxiety, relieve stress, strength the user's immune system, and the like. Camphene has antifungal benefits against certain fungi when combined with sage oil. Fenchone has anti-inflammatory and antimicrobial properties, increases collagen synthesis, and decreases the number of inflammatory cells during wound healing. Linalool is purported to treat conditions such as anxiety and depression and can help to combat insomnia.

In various embodiments, male users further comprise users who will generally benefit from a chemical composition that is different from the chemical composition intended for female only users. For example, users who are biosimilar to male users because they are undergoing or have completed gene therapy, hormone therapy, transgender treatments, and the like.

In contrast to the chemical composition of TABLE 1 which has been tailored for enhancement of a male only user's vaping experience, the chemical composition of TABLE 2 has been tailored for enhancement of a female user's vaping experience. That is, TABLE 2 describes a second enhanced chemical composition intended for a female only user.

TABLE 2

| | | | | Percentage | |
|---|---|---|---|---|---|
| | | Variance | | Variance | |
| Ingredient | MG/Gram | (+/−) | Percentage | (+/−) | Effect |
| Hexahydro | 800 | 160 | 80.00% | 16.00% | Promotes |
| Cannabinol | | | | | Relaxation, |
| | | | | | Heighten |
| | | | | | Sensitivity to |
| | | | | | Touch, and |
| | | | | | Increased Intensity |
| | | | | | of Feelings |
| Hexahydrothymol | 3.706 | 0.741 | 0.37% | 0.07% | Ergogenic - may |
| | | | | | enhance physical |
| | | | | | performance and |
| | | | | | stamina. |
| Trans- | 3.468 | 0.694 | 0.35% | 0.07% | Reduce Stress |
| Caryophyllene | | | | | |
| (R)-(+)-Limonene | 3.346 | 0.669 | 0.33% | 0.07% | Elevate Mood by |
| | | | | | Increasing |
| | | | | | Serotonin, |
| | | | | | Dopamine, |
| | | | | | Norepinephrine, |
| | | | | | and GABA |
| | | | | | concentrations in |
| | | | | | the Brain |
| Linalool | 3.016 | 0.603 | 0.30% | 0.06% | Induce Body and |
| | | | | | Mind Relaxation - |
| | | | | | May increase |
| | | | | | Blood Flow |
| | | | | | through |
| | | | | | Vasodilation |
| 3-Carene | 3.006 | 0.601 | 0.30% | 0.06% | Improve Mental |
| | | | | | State -May |
| | | | | | Improve |
| | | | | | Attentiveness |
| Alpha-Pinene | 2.915 | 0.583 | 0.29% | 0.06% | Increase Feeling |
| | | | | | of Energy |
| Eucalyptol | 2.803 | 0.561 | 0.28% | 0.06% | Promote Calmness |
| Beta-Pinene | 2.602 | 0.52 | 0.26% | 0.05% | Increase Alertness |
| beta-Myrcene | 2.325 | 0.465 | 0.23% | 0.05% | Enhance |
| | | | | | Bioavailability |
| Geraniol | 2.238 | 0.448 | 0.22% | 0.04% | Enhance |
| | | | | | Relaxation - |
| | | | | | Vasodilation |
| Nerol | 1.148 | 0.23 | 0.11% | 0.02% | Vasodilation May |
| | | | | | increase Blood |
| | | | | | Flow through |
| | | | | | Vasodilation |
| Terpineol | 1.103 | 0.221 | 0.11% | 0.02% | Reduce Anxiety - |
| | | | | | May increase |
| | | | | | Blood Flow |
| | | | | | through |
| | | | | | Vasodilation |
| Fenchone | 0.384 | 0.077 | 0.04% | 0.01% | Aromatic Balance |
| Caryophyllene | 0.174 | 0.035 | 0.02% | 0.00% | CB2 Receptor |
| oxide | | | | | Agonist for |
| | | | | | Enhancement of |
| | | | | | Entourage Effect |
| alpha-Humulene | 0.114 | 0.023 | 0.01% | 0.00% | Enhance |
| | | | | | Bioavailability |
| Camphene | 0.057 | 0.011 | 0.01% | 0.00% | Reduce Stress |

In various embodiments, female users further comprise users who will generally benefit from chemical composition that is different from the chemical composition intended for male only users. For example, users who are biosimilar to female users because they are undergoing or have completed gene therapy, hormone therapy, transgender treatments, and the like.

In some embodiments, the amount of active ingredient in the chemical composition is different for male users and female users. For example, the amount of an active ingredient is greater for a male user than for a female user. Alternatively, the amount of an active ingredient is smaller for a male user than for a female user. Using the list of active ingredients from TABLE 1 and TABLE 2 as an illustrative example, the amount of active ingredient for limonene, a substance shown to possess antioxidant, anti-inflammatory, anti-stress, and possibly possessing disease-preventing properties, is greater for male only users than it is for female only users. Likewise, the amount of active ingredient for eucalyptol, a substance shown to have antioxidant and anti-inflammatory properties, and caryophyllene oxide a substance shown to have anti-inflammatory and local anesthetic properties, is also greater for male only users than it is for female only users.

In various embodiments, one or more active ingredients may be added or removed from TABLE 1 and/or TABLE 2, for the purpose of increasing or decreasing the quality, magnitude, and/or duration of the effects per the corresponding active ingredient, subject to modification.

In some embodiments, one or more active ingredients may be added to or removed from TABLE 1 and/or TABLE 2, at any time to accommodate the preference of the user and regional requirements, ethnicity requirements, culture requirements, judicial requirements, and the like.

In some embodiments, the one or more active ingredients may be removed from TABLE 1 and/or TABLE 2 to accommodate possible adverse reactions, allergic or otherwise of an individual user or users. In some embodiments, one or more active ingredients may be added and/or removed from the chemical composition in real-time by a vaping device.

In TABLE 1 and TABLE 2, the “Ingredient” column identifies the active ingredient. The “Mg/G” column identifies the amount of active ingredient in milligrams. The “Variance (+/−)” column identifies the magnitude of the possible deviation of the ingredient amount that will generally maintain the desired effect in the chemical composition. The “Percentage” column identifies the ratio or proportion of the active ingredient with respect to the chemical composition. The “Percentage Variance (+/−)” column identifies the magnitude of the possible deviation of the ratio, or proportion of the active ingredient with respect to the chemical composition that will generally maintain the desired effect. Lastly, and the “Effect” column identifies at least some of the desired effect(s) that the active ingredient adds to the chemical composition.

In various embodiments, the composition of the chemical composition is different for male users and female users. That is, the composition of the substance may be specifically tailored for a male user. For example, an active ingredient may be added to the composition of the chemical composition for a male user that would not normally be used if the user was female. Alternatively, an active ingredient may be omitted from the composition of the chemical composition for a male user that would normally be included if the user was female.

In various embodiments, one or more active ingredients may be added, removed, or modified from TABLE 1 and/or TABLE 2, for the purpose of increasing or decreasing the quality, magnitude, and/or duration of the effects per the corresponding ingredients subject to modification.

In some embodiments, the amount of active ingredient is substantially the same for both male only users and female only users. Using the list of active ingredients from TABLE 1 and TABLE 2 as an illustrative example, the amount of active ingredient for alpha-humulene, a substance thought have appetite-suppressing effects and promote weight loss, is substantially the same for both male users and female users.

In various embodiments, the composition of the chemical composition is different if both male users and female users are the intended recipients. For example, if the chemical composition is intended to be shared by both male and female users. That is, the composition of the chemical composition that is intended to be shared by both male and female users is different from the composition intended for male only users or for female only users.

In various embodiments, the active ingredient may be intended for both male only users and female only users, however the amount of active ingredient is smaller for the male only user than it is for female only user. In some embodiments, the amount of active ingredient for the male only user is slightly smaller than the amount of active ingredient for the female only user. In other embodiments, the amount of active ingredient for the male only user is significantly smaller than the amount of active ingredient for the female only user. Using the list of active ingredients from TABLE 1 and TABLE 2 as an illustrative example, the amount of active ingredient for Hexahydrothymol a substance purported to have benefits including but not limited to antiseptic, anti-inflammatory, and anti-spasmodic properties is less for male only users than it is for female only users. Hexahydrothymol is a colorless, crystalline, slightly water-soluble alcohol C₁₀H₂₀O that can be synthesized or obtained from peppermint oil that is used in medicines to treat cold and nasal disorders due to Hexahydrothymol's cooling effect on the mucous membranes. Other active ingredients where the amount of active ingredient in the chemical composition for a male only user is less than the amount for a female only user may include, but is not limited to, one or more of: 3-Carene, alpha-Pinene, beta-Pinene, Camphene, Fenchone, Geraniol, trans-Caryophyllene, Linalool, Nerol, and Terpineol.

In various embodiments, the active ingredient may be intended for both male only users and female only users, however the amount of active ingredient is smaller for the female only user than it is for male only user. In some embodiments, the amount of active ingredient for the female only user is slightly smaller than the amount of active ingredient for the male only user. In other embodiments, the amount of active ingredient for the female only user is significantly smaller than the amount of active ingredient for the male only user. Using the list of active ingredients from TABLE 1 and TABLE 2 as an illustrative example, the amount of active ingredient for limonene, a substance shown to possess antioxidant, anti-inflammatory, anti-stress, and possibly possessing disease-preventing properties, is smaller for female only users than it is for male only users. Likewise, the amount of active ingredient for eucalyptol, a substance shown to have antioxidant and anti-inflammatory properties, and caryophyllene oxide a substance shown to have anti-inflammatory and local anesthetic properties, is also smaller for female only users than it is for male only users.

In various embodiments, the chemical composition further comprises one or more inactive compounds that serve as a medium for the active ingredient. That is, the chemical composition comprises one or more pharmaceutically acceptable additives, carriers, excipients, and the like. For example, inactive compounds that serve as a medium for the active ingredient may include flavoring agents, organic solvents, pH adjusting agents, sweetening agents, volumizers, water, and the like.

In various embodiments, the chemical composition of excipients, additives, solvents, and the like, is substantially the same, within the variance of the other active ingredients, for both male only users and female only users. For example, using the list of ingredients from TABLE 1 and TABLE 2 as an illustrative example, the volume of Hexahydro cannabinol is substantially the same for both male users and female users. Hexahydro cannabinol or “HHC,” is a cannabinoid, which are naturally occurring compounds found cannabis plants. The most commonly known cannabinoids are Delta-9 THC and cannabidiol (CBD). THC is the ingredient in marijuana that promotes euphoria or makes you feel high. Cannabidiol is the ingredient in many CBD products, and is purported to help with anxiety, depression, pain and soreness, sleep problems, and the like.

In various embodiments, a solvent, such as alcohol, may be used in liquid vaping chemical compositions. Examples, of alcohol based solvents include ethanol, ethyl acetate, glycerin, a colorless, odorless liquid having a slightly sweet taste, propylene glycol CH₃CHCH₂OH, a viscous, colorless, and nearly odorless liquid with a faintly sweet taste, and the like. In some embodiments, the chemical composition comprises a combination of two or solvents. The amount of solvent in the chemical composition varies but is generally equal to or greater than about 50% of the chemical composition by weight. In some instances, the amount of solvent in the chemical composition is equal to or greater than about 80% of the chemical composition by weight.

In some embodiments, the solvent may be water. The amount of water in the chemical composition varies, but is generally between less than, or equal to about 10% of the chemical composition by weight. For example, the amount of water in the chemical composition may be between about 0.1% and about 5% of the chemical composition by weight.

In various embodiments, the pH level of the chemical composition is adjusted by using acidifying and alkalizing agents. Acidifying agents are used to lower the pH level of the chemical composition, whereas alkalizing agents are used to increase the pH level of the chemical composition. In some embodiments, the pH level of the chemical composition may be modified by the addition of a pH adjusting agent like an inorganic acid. For example, the pH adjusting agent may be one or more inorganic acids such as hydrobromic acid, hydrochloric acid, phosphoric acid, sulfuric acid, and the like. In some embodiments, the pH level of the chemical composition may be modified by the addition of a pH adjusting agent like an organic acid. For example, the pH adjusting agent may be one or more organic acids, such as acetic acid, ascorbic acid, citric acid, formic acid, fumaric acid, lactic acid, maleic acid, malic acid, succinic acid, propionic acid, tartaric acid, and the like.

In various embodiments, two or more pH adjusting agents are added to the chemical composition. In some embodiments, only one pH adjusting agent is added to the chemical composition. Some pH adjusting agents (acids) may have additional properties that make them attractive for inclusion in the chemical composition. For example, some acids have antioxidant properties and/or flavor properties that make them attractive for inclusion in the chemical composition.

In some embodiments, the pH level of the chemical composition may be modified by the addition of a pH adjusting agent like an alkali. An alkali is a basic, ionic salt of an alkali metal or an alkaline earth metal. For example, the pH adjusting agent may be an alkali metal hydroxide, alkali metal carbonate, and the like. In some embodiments, the alkali ion in the carbonate or alkali metal hydroxide is sodium. The pH level of the chemical composition is adjusted by the addition of pH adjusting agents until the desired pH level is obtained and depends, at least in part, on the starting pH level of the chemical composition.

In some embodiments, the chemical composition further comprises a pharmacologically or pharmaceutically acceptable excipient. Pharmaceutical or pharmaceutically excipients are compounds that are included in a pharmaceutical dosage form not for their direct therapeutic action, but to aid the manufacturing process, to protect, support or enhance the stability of the chemical composition, or for bioavailability or patient acceptability. For example, the pharmacologically or pharmaceutically acceptable excipient may be one or more of an ascorbic acid, vitamins or provitamins, such as vitamin A, vitamin E, and the like.

In some embodiments, one or more preservatives may be added to the chemical composition. For example, the preservative is one or more of benzoic acid, benzalkonium chloride, sodium benzoate, and the like.

In some embodiments, one or more flavoring agents and/or sweeteners may be added to the chemical composition. For example, the flavoring agent and/or sweetener is one or more of cinnamon, eucalyptus, menthol, peppermint, propolis, spearmint, wintergreen, and the like. The amount of flavoring in the chemical composition varies but is generally between less than or equal to about 50% by weight of the chemical composition. For example, the amount of flavoring in the chemical composition may be between about 0.1% and about 15% by weight.

The foregoing description has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

I/We claim:

1. A chemical composition suitable for use in a vaping device, the chemical composition comprising:
a constituent group of ingredients, wherein the chemical composition of the constituent group of ingredients is operable for use by a male only user, is operable for use by a female only user, and operable to be shared by both a male user and a female user, and wherein the constituent group of ingredients is different for each of the male only user, the female only user, and that intended to be shared by both the male user and the female user.

2. The chemical composition of claim 1, wherein the chemical composition of the constituent group of ingredients intended to be shared by both a male user and a female user is operable to produce two or more different effects.

3. The chemical composition of claim 1, wherein chemical composition of the constituent group of ingredients intended to be shared by both a male user and a female user has two or more different purposes, and wherein the two or more different purposes comprises at least a sexual activity.

4. The chemical composition of claim 1, wherein the chemical composition of the constituent group of ingredients intended to be shared by both a male user and a female user is operable to be different from the chemical composition intended to be used by a male only user and the chemical composition intended to be used by a female only user.

5. The chemical composition of claim 1, wherein the chemical composition of the constituent group of ingredients intended for a female only user, and wherein the chemical composition intended for a female only user comprises one or more active ingredient(s) that the chemical composition intended for a male only user does not contain.

6. The chemical composition of claim 1, wherein the chemical composition of the constituent group of ingredients intended for a male only user is different from the composition of the chemical composition intended for a female only user, and wherein the composition of the chemical composition intended for the male only user comprises a greater amount of an active ingredient than the chemical composition intended for a female only user.

7. The chemical composition of claim 6, wherein the chemical composition of the constituent group of ingredients intended for the male only user comprises one or more active ingredient(s) that the chemical composition intended for the female only user does not contain.

8. The chemical composition of claim 6, wherein the chemical composition of the constituent group of ingredients having attributes tailored for the male user is preferred over the chemical composition having attributes tailored for the female user for a user who is biosimilar to a male user.

9. The chemical composition of claim 5, wherein the chemical composition of the constituent group of ingredients having attributes tailored for the female user is preferred over the chemical composition having attributes tailored for the male user for a user who is biosimilar to a female user.

10. The chemical composition of claim 1, wherein the chemical composition of the constituent group of ingredients comprises an active ingredient, wherein the active ingredient is used by both a male only user and a female only user, wherein an amount of the active ingredient used by the male only user is less than the amount of active ingredient used by the female only user, and wherein the active ingredient is one or more of 3-Carene, alpha-Pinene, beta-Pinene, Camphene, Fenchone, Geraniol, Hexahydrothymol, trans-Caryophyllene, Linalool, Nerol, and Terpineol.

11. The chemical composition of claim 1, wherein the chemical composition of the constituent group of ingredients further comprises at least one or more preservative, additive, and pH adjusting agent, and wherein the pH adjusting agent is an alkali.

12. The chemical composition of claim 1, wherein the chemical composition of the constituent group of ingredients further comprises a pharmaceutically excipient.

13. The chemical composition of claim 1, wherein, the chemical composition of the constituent group of ingredients further comprises one or more active ingredients.

14. The chemical composition of claim 1, wherein the chemical composition of the constituent group of ingredients further comprises a pH adjusting agent, and wherein the pH adjusting agent is one or more of an organic acid and an inorganic acid.

15. The chemical composition of claim 1, wherein the chemical composition of the constituent group of ingredients further comprises a solvent, and wherein the solvent is one or more of an alcohol based solvent and a water based solvent.

16. The chemical composition of claim 1, wherein the chemical composition of the constituent group of ingredients further comprises a cannabinol based product, and wherein the cannabinol based product is hexahydro cannabinol.

17. The chemical composition of claim 1, wherein the constituent group of ingredients comprises a limonene based product, and wherein the limonene based product is one or more of: 3-Carene, Alpha-Pinene, Alpha-Humulene, Ashwagandha, Beta-Myrcene, Beta-Pinene, Camphene, Caryophyllene oxide, Eucalyptol, Fenugreek seed extract, Fenchone, Gamma-Terpinene, Geraniol, Geranyl acetate, Hexahydrothymol, horny goat weed extract trans-Caryophyllene, (R)-(+)-Limonene, Linalool, Nerol Terpineol, Tribulus Terrestre's, Terpinolene, and Maca root extract.

18. A vaping device having a chemical composition suitable for use in the vaping device, the chemical composition comprising:
a constituent group of ingredients, wherein the chemical composition of the constituent group of ingredients is operable for use by a male only user, is operable for use by a female only user, and operable to be shared by both a male user and a female user, and wherein the constituent group of ingredients is different each of the male only user, the female only user, and that intended to be shared by both the male user and the female user.
